(19) United States
(12) Patent Application Publication
(10) Pub. No.: US 2018/0021064 A1
(43) Pub. Date: Jan. 25, 2018

(54) Suction Tube for Abortion and Using Method Thereof

(71) Applicant: Zhejiang Shanshi Biological Medical (Shangqiu) Co., Ltd., Shangqiu (CN)

(72) Inventor: Junjie Bian, Shangqiu (CN)

(21) Appl. No.: 15/653,795

(22) Filed: Jul. 19, 2017

(30) Foreign Application Priority Data
Jul. 22, 2016 (CN) 2016105836589
Jul. 22, 2016 (CN) 2016207788443

(51) Int. Cl. A61B 17/42; A61M 1/16; A61M 1/00

(52) U.S. Cl. CPC A61B 17/42; A61M 1/0001; A61M 1/1621; A61B 2017/4216; A61B 2017/306

(57) ABSTRACT

A suction tube for abortion is provided in the invention, including a tube body, wherein the tube body includes a first cavity and a second cavity, the first cavity is a suction cavity, having an inlet for sucking from the body and an outlet for discharging to the outside of the body; and the second cavity also includes an inlet for connecting with the inside body and an outlet for connecting with the outside, wherein the outlet is connected with the fluid in the external environment through a first conduit and a second conduit, the first conduit can only connect with the fluid in a one-way manner from the outside to the second cavity, and the second conduit can only connect with the fluid in a one-way manner from the second cavity to the outside. When in use, the invention can prevent the uterine blood from reversely flowing in the induced abortion, and prevent against endometriosis and infertility; reduce the muscular tension of the uterine wall in the induced abortion, and reduce bleeding and pain.

### RELATED APPLICATIONS

This application claims priority to CN 2016207788443, filed Jul. 22, 2016 and CN 2016105836589, filed Jul. 22, 2016, each of which is incorporated herein in their entirety.

### FIELD OF THE INVENTION

The invention relates to a medical device, in particular, to a suction tube for abortion.

### BACKGROUND

The existing suction tube for abortion is a single-cavity tube, and its function is single. When the patient puts in or pulls out the suction tube, it will be easy to cause uterine contraction. At that time, the pressure inside the uterine cavity will increase rapidly, and gas or liquid will form backflushing, which is very easy to develop endometriosis, causing infertility. At the same time, it will excite the vagus nerve, and develop the artificial abortion syndrome. During suction, it is easy to cause negative pressure. If it is not discharged in time, the blood vessels and the pressure difference in the uterine cavity will easily become larger, the muscular tension of the uterine wall will then be increased, and the patient may bleed excessively and easily. This not only increases the pain and the risks of the patient, but also extends the time of operation.

In the prior art, the inventor once applied for another three-cavity suction tube for abortion (ZL2012101560923). Of course, many drawbacks of the single-cavity suction tube for abortion are overcome, but this design still has some deficiencies, which are mainly reflected in the following aspects: the production and manufacturing cost for three cavities is high, and the production technology is complex; in addition, when the cross section of the suction tube is the same, and as there are three cavities, with two independent cavities occupied respectively by the pressure reducing cavity and the negative pressure control cavity, the flow of the suction cavity will certainly be reduced, increasing the difficulties in suction and the number of times of suction, thus causing a lot of discomfort of the patient. Therefore, it is necessary to improve the existing disclosed three-cavity suction tube for abortion, so as to overcome the technical defects of the existing product.

### SUMMARY

To overcome the drawbacks of the prior art, the invention provides a two-cavity suction tube for abortion, with one cavity shared by the pressure reducing cavity and the negative pressure control cavity, increasing the flow of the suction cavity, and reducing the discomfort and pain of the patient.

On the one hand, the invention provides a suction tube for abortion, including a tube body, wherein the tube body includes a first cavity and a second cavity, wherein the first cavity is a suction cavity, having an inlet for sucking from the body and an outlet for discharging to the outside of the body; and the second cavity also includes an inlet for connecting with the inside body and an outlet for connecting with the outside, wherein the outlet is connected with the fluid in the external environment through a first conduit and a second conduit, wherein the first conduit can only connect with the fluid in a one-way manner from the outside to the second cavity, and the second conduit can only connect with the fluid in a one-way manner from the second cavity to the outside.

In a preferred embodiment, the inlet of the first cavity and the inlet of the second cavity can be connected with the uterus phase fluid.

In a preferred embodiment, wherein a dustproof filter layer is provided in the first conduit.

In a preferred embodiment, wherein on the cross section of the tube body, the first cavity is on one side, and the second cavity is on the other side.

In a preferred embodiment, wherein the inlet of the first cavity and the inlet of the second cavity are on two sides of the tube body, on the end connecting with the uterus in the body; the first conduit and the second conduit are on the other end of the tube body, and connect with the outside.

In a preferred embodiment, wherein the outlet of the first cavity of the suction tube for abortion connects with the suction device, and the first conduit connects with another gas conveying device.

In a preferred embodiment, wherein the outlet of the first cavity of the suction tube for abortion connects with the fluid connection of the suction device through a handle, and the first conduit connects with the fluid connection of another gas conveying device through a handle.

In a preferred embodiment, wherein a visual guiding device is provided, wherein the visual guiding device is provided near the inlet of the first cavity, and the first conduit and the second conduit are in the rear of the visual guiding device.

In a preferred embodiment, wherein the visual guiding device includes a pin-type camera and a light source.

In a preferred embodiment, wherein the fluid includes gas, liquid, or a mixture of liquid and gas.

In a preferred embodiment, the first conduit is a pressure reducing outlet pipe, and the second conduit is a negative pressure inlet pipe.

The handle includes a major joint for connecting with the suction tube body, an inlet for connecting with the suction outlet of the suction cavity, and a circulation channel between them; and the handle further includes a one-way outlet and a one-way inlet; the one-way inlet connects with the negative pressure inlet pipe, wherein a one-way air inlet valve is provided, and the one-way outlet connects with the pressure reducing outlet pipe, wherein a one-way air outlet valve is provided.

A dustproof filter layer is provided on the one-way inlet.

In the invention, a visual guiding device can be further provided. The visual guiding device is provided at the head of the suction tube body, the pressure reducing inlet and the negative pressure control air outlet are in the rear of the visual guiding device.

The visual guiding device includes a pin-type camera and a light source.

On the other hand, the invention provides a method of using the suction tube for abortion, and the method includes:

A suction tube for abortion is provided, including a tube body, wherein the tube body includes a first suction cavity and a second adjustment cavity, wherein the first cavity is a suction cavity, having an inlet for sucking from the body and an outlet for discharging to the outside of the body; and the second cavity also includes an inlet for connecting with the inside body and an outlet for connecting with the outside, wherein the outlet is connected with the fluid in the external environment through a first conduit and a second conduit, wherein the first conduit can only connect with the fluid in a one-way manner from the outside to the second cavity, and the second conduit can only connect with the fluid in a one-way manner from the second cavity to the outside; wherein a gas pressure sensor is provided respectively in the suction cavity and the adjustment cavity, and the sensors connect with the controller and the host machine, wherein the controller connects with the device for extracting air from the suction cavity, and the air inlet device for letting gas enter the first conduit;

When the inlets of the first suction cavity and the second adjustment cavity of the suction tube for abortion enter the uterus, the host machine automatically starts a mode. In this mode, the initial value of the gas pressure sensor in the suction cavity 10 should be maintained less than that in the adjustment cavity 20. Preferably, the initial value of the gas pressure sensor in the suction cavity 10 should be a half of that in the adjustment cavity 20.

In a preferred embodiment, the inlet of the first cavity and the inlet of the second cavity can be connected with the uterus phase fluid.

In a preferred embodiment, wherein a dustproof filter layer is provided in the first conduit.

In a preferred embodiment, wherein on the cross section of the tube body, the first cavity is on one side, and the second cavity is on the other side.

In a preferred embodiment, wherein the inlet of the first cavity and the inlet of the second cavity are on two sides of the tube body, on the end connecting with the uterus in the body; the first conduit and the second conduit are on the other end of the tube body, and connect with the outside.

In a preferred embodiment, wherein the outlet of the first cavity of the suction tube for abortion connects with the suction device, and the first conduit connects with another gas conveying device.

In a preferred embodiment, wherein the outlet of the first cavity of the suction tube for abortion connects with the fluid connection of the suction device through a handle, and the first conduit connects with the fluid connection of another gas conveying device through a handle.

In a preferred embodiment, wherein a visual guiding device is provided, wherein the visual guiding device is provided near the inlet of the first cavity, and the first conduit and the second conduit are in the rear of the visual guiding device.

In a preferred embodiment, wherein the visual guiding device includes a pin-type camera and a light source.

In a preferred embodiment, wherein the fluid includes gas, liquid, or a mixture of liquid and gas.

In a preferred embodiment, the first conduit is a pressure reducing outlet pipe, and the second conduit is a negative pressure inlet pipe.

### Beneficial Effects

Due to the adoption of the above technical solutions, the main difference between the existing suction tube for abortion and the invention is that: when the invention is used, once the systolic pressure or negative pressure occurs in the uterine cavity, the pressure difference can be adjusted in time through the pressure reducing cavity and the negative pressure control cavity, which can prevent the uterine blood from reversely flowing in the induced abortion, and prevent against endometriosis and infertility; reduce the muscular tension of the uterine wall in the induced abortion, and reduce bleeding and pain; at the same time, the pressure reducing cavity and the negative pressure control cavity work in a one-way manner, with one in and one out, so the discharged grimy blood will not flow backwards, avoiding cross infection; the pressure regulating of the inflowing air can, through the filter layer, prevent unclean air in the operation room from directly entering the uterine cavity, thus reducing the infection probability of uterine cavity. And the provision of the visual guiding device further increases the intuition and accuracy rate of the operation (directly towards the embryonic tissue), and it does not influence the normal operation of the three cavities; thus, the best treatment method (minimally invasive therapy has the minimal trauma) can be obtained by using the shortest treatment time and the most accurate target treatment means, so as to reach the best treatment effect.

In addition, as two cavities are adopted for control, and when the cross section is the same, the cross sectional area of the suction cavity can be increased effectively, and the circulation volume of the fluid sucked per unit time can be increased, thus it can effectively avoid blocking the suction cavity. Moreover, this application adopts an environment under which the pressure of the two different cavities is controlled automatically, so as to realize automatic adjustment. In this way, when the suction tube is adopted to suck the fertilized embryo in the uterus, the damages to the uterus can be effectively reduced, and the degree of comfort of suction can be increased, minimizing the degree of uterine contraction.

### BRIEF DESCRIPTION OF THE DRAWINGS

FIG. 1 is a sectional view according to Example 1 of the invention.

FIG. 2 is a longitudinal sectional view according to Example 1 of the invention.

FIG. 3 is a schematic diagram of the operating process of abortion according to Example 1 of the invention.

FIG. 4 is a structural diagram of the conduit (including a pressure sensor) according to another specific embodiment of the invention.

### DETAILED DESCRIPTION

### Example 1

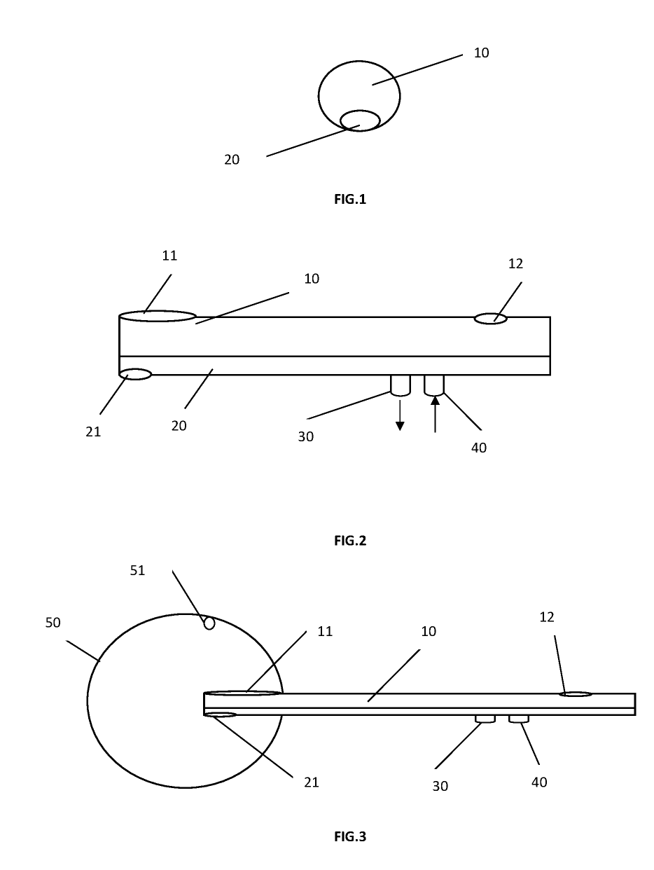

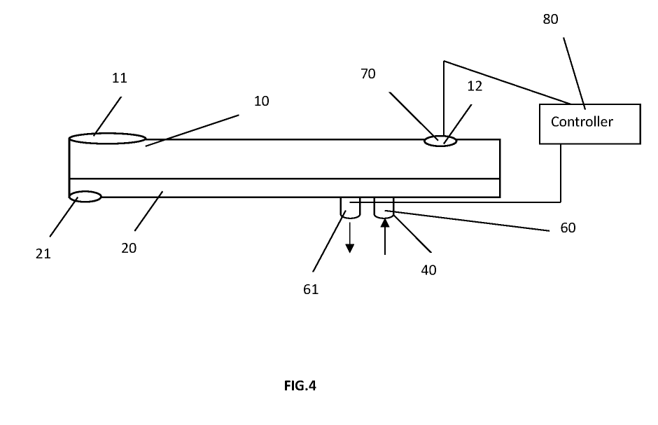

Referring to the attached FIGS. 1, 2 and 3, the invention includes a suction tube body, wherein the tail of the tube body stretches into the handle (not shown) and connects with it, and the tube body 1 is divided into two tubular cavities extending along the lengthwise direction of the tube body; wherein the first cavity is a suction cavity 10, having a suction inlet 11 and a suction outlet 12; and the second cavity is a adjustment cavity 20, having an inlet 21; on one end of the first cavity, there are two pipes connected with the fluid of the second cavity, wherein one is the negative pressure control pipe 30 and the other is the pressure reducing control pipe 40.

In addition, both the first conduit and the second conduit are provided with a one-way valve, or wherein the air inlet pipe is provide with a one-way valve, and gas can only enter and can not come out of the pipe. For example, the negative pressure control pipe 30 is an air inlet pipe, and gas can only enter and can not come out of this pipe. Or, the pressure reducing control pipe 40 is an air outlet pipe, and gas can only be discharged from this pipe and can not enter this pipe. A dustproof filter layer is provided on the one-way inlet, or an air filter layer is installed in the one-way inlet pipe, which can filter the bacteria and viruses in the air, making the inflowing air clean.

The two cavities 10 and 20 in the tube body 1 can be formed by dividing the tube body by the diaphragm in the tube.

On the cross section of tube body, the pressure control cavity 20 is on one side and the suction cavity 10 is on the other side. In this way, sufficient area for installation can be provided, which is convenient for the installation of the visual guiding device, the normal operation of the two cavities will not be influenced, and it is convenient for the connection and arrangement of wires of the visual guiding device.

Before operating, first install the tube body provided in the invention on the handle, on which there is a through hole for mutual fluid connection with the outlet of the suction cavity 12, and through this through hole, it can be connected with the suction device. In addition, on one end of the adjustment cavity 20, respectively corresponding to the inlet hole and outlet hole on the handle, wherein the one-way inlet pipe 30 connects with the one-way air inlet device, and the one-way outlet pipe 40 connects with the outside.

When it is necessary to suck the tissues in some tracheas such as intestines and stomach, uterus 50, or fall off the embryo 51, now we use labor induction to describe how to operate this tube. When it is necessary to enter the uterus, you can stretch one end of the tube, that is, the suction cavity with the inlet 11 and the adjustment cavity with the inlet 12 into the uterus. At this time, due to manual operation, a foreign body may more or less touch the uterine wall as it is stretched into the uterus, and due to the conditioned reflex of the uterus, this will cause uterine contraction or tension. When the uterus is under a natural condition, its internal fluid or pressure is constant, and the volume is also substantially constant. However, as a foreign body enters the uterus suddenly, a uterine contraction will be caused, and the volume will be reduced, the internal pressure will be increased. At this time, as the internal pressure is increased, the patient will feel uncomfortable (although anesthetics are given), and the uterus may still be damaged. The redundant gas or mixture of gas and blood will enter the adjustment cavity along the inlet 21 of the adjustment cavity 20, and as the air pressure in the adjustment cavity is increased, they will be discharged to the outside along the air outlet pipe 40.

When discovering the embryo, it is necessary to increase the pressure of the suction cavity, so as to let the inlet 11 of the suction cavity correspond to the embryo, and let the embryo fall off from the surface of the uterus. At this time, add a suction device, to suck the gas of the suction tube, so as to increase the adhesive force of the embryo to the inlet 11, and thus to let the embryo fall off from the endometrium smoothly. Actually, adding the attractive force of the suction cavity is to let the pressure of the suction cavity become negative, so as to allow the gas in the uterus to be sucked. In fact, the pressure of the uterus is also negative. So, it is necessary to open the air inlet pipe, and through the air inlet device, one-way air inlet pipe 30 and adjustment cavity 20, to let the outside gas enter the uterus via the inlet 21, thus relieving the negative pressure of the uterus.

Although the above method is practicable, it is not very satisfactory in actual operation. This is because the air extractor extracts air via the suction cavity, and the air inlet device puts air into the uterus via the adjustment cavity in a one-way manner, these two devices work separately, and one can not well cooperate with the other. Particularly, it is necessary to adjust the admission speed or the extraction speed of each device (such as air inlet device and air extractor), thus a professional test is needed. In addition, for each subject, their tolerating limitations are different, the degree of embryo attaching to the uterine wall, the size of the uterus and the thickness of the uterine wall are different too. This requires a coordinated collaboration between gas conveying devices (such as air inlet device and air extractor) that operate via pipes, so as to maximize the degree of comfort.

In some embodiments, referring to FIG. 4, an air pressure inductive sensor (air pressure sensor) is provided in the air inlet pipe, for example, a gas pressure sensor 70 is provided in the suction cavity, and the sensor connects with the controller 80 and the host machine (not shown), at the same time, the air inlet device and the air extractor (not shown) respectively connect with the controller. When this tube is used, and the suction is performed through the suction cavity, the pressure sensor 70 in the suction cavity will obtain the pressure data, and the pressure will be reduced, showing that the pressure in the uterus is the same as that of the suction cavity. At this time, the signal will be sent to the controller 80 and the host machine, then the host machine will give a command, to start the air inlet device, and let the gas enter the uterus through the one-way air inlet pipe 30, thus increasing the pressure in the uterus.

In some preferred embodiments, a pressure sensor can be provided on the visual guiding device, for detecting the pressure in the uterus from time to time. More preferably, a pressure sensor is provided respectively on the air inlet pipe and the air outlet pipe of the adjustment cavity 20, for monitoring the air pressure in the adjustment cavity 20 from time to time. After we perform clinical experiments for more than once, we find that, when a gas pressure sensor 70 is provided in the suction cavity 10 and two pressure sensors 61 and 60 are respectively provided in the adjustment cavity 20, let the initial value of the sensor in the suction cavity 10 be less than that of the sensor in the adjustment cavity 20 (if we let the former one be a half of the latter one, we will obtain the best effect), and such values are opened automatically or manually only after the tube enters the uterus. In this way, let the pressure of the suction cavity 10 always be less than that in the adjustment cavity 20, making the inlet 11 of the suction cavity 10 easier to suck the embryo, and making the embryo easier to fall off from the endometrium. This can minimize the damages to the uterus, and maximize the degree of comfort of the patient. According to 150 cases of clinical experiments, compared with traditional abortion operation (using the tube disclosed in ZL2012101560923), the degree of comfort of the patient by using this tube increases by over 40%, with no uterus injury case. When the tube disclosed in ZL2012101560923 is used for induced abortion, approximately 30 to 45 minutes will be spent for a case, while when the automatic-adjustment air pressure sensor provided in the invention is used, approximately 10 to 15 minutes will be spent for a case. The time is nearly reduced by a half, and thus the operative risk is reduced. This is because, the longer the operation lasts, the higher the injury risk will be, and the more unsafe the operation will be.

1. A method of using suction tube for abortion, comprising:
providing a suction tube for abortion, comprising a tube body, wherein the tube body comprises a first cavity and a second cavity, the first cavity is a suction cavity, having an inlet for sucking from the body and an outlet for discharging to the outside of the body; and the second cavity also comprises an inlet for connecting with the inside body and an outlet for connecting with the outside, wherein the outlet is connected with the fluid in the external environment through a first conduit and a second conduit, wherein the first conduit can only connect with the fluid in a one-way manner from the outside to the second cavity, and the second conduit can only connect with the fluid in a one-way manner from the second cavity to the outside; wherein a gas pressure sensor is provided respectively in the suction cavity and the adjustment cavity, and the sensors connect with the controller and the host machine, wherein the controller connects with the device for extracting air from the suction cavity, and the air inlet device for letting gas enter the first conduit;
When the inlets of the first suction cavity and the second adjustment cavity of the suction tube for abortion enter the uterus, the host machine automatically starts a mode, in this mode, the initial value of the gas pressure sensor in the suction cavity should be maintained less than that in the adjustment cavity.

2. The method according to claim 1, wherein an inlet of the first cavity and an inlet of the second cavity can be connected with the uterus phase fluid.

3. The method according to claim 1, wherein a dustproof filter layer is provided in the first conduit.

4. The method according to claim 1, wherein the inlet of the first cavity and the inlet of the second cavity are on two sides of the tube body, on the end connecting with the uterus in the body; the first conduit and the second conduit are on the other end of the tube body, and connect with the outside.

5. The method according to claim 1, wherein the outlet of the first cavity of the suction tube for abortion connects with the suction device, and the first conduit connects with another gas conveying device.

6. The method according to claim 1, wherein the initial value of the gas pressure sensor in the suction cavity is a half of that in the adjustment cavity.

7. A suction tube for abortion, including a tube body, wherein the tube body includes a first cavity and a second cavity, wherein the first cavity is a suction cavity, having an inlet for sucking from the body and an outlet for discharging to the outside of the body; and the second cavity also includes an inlet for connecting with the inside body and an outlet for connecting with the outside, wherein the outlet is connected with the fluid in the external environment through a first conduit and a second conduit, wherein the first conduit can only connect with the fluid in a one-way manner from the outside to the second cavity, and the second conduit can only connect with the fluid in a one-way manner from the second cavity to the outside.

8. The suction tube to claim 7, wherein, the inlet of the first cavity and the inlet of the second cavity can be connected with the uterus phase fluid.

9. The suction tube to claim 8, wherein, a dustproof filter layer is provided in the first conduit and wherein on the cross section of the tube body, the first cavity is on one side, and the second cavity is on the other side.

10. The suction tube to claim 9, wherein the inlet of the first cavity and the inlet of the second cavity are on two sides of the tube body, on the end connecting with the uterus in the body; the first conduit and the second conduit are on the other end of the tube body, and connect with the outside.

11. The suction tube to claim 10, wherein the outlet of the first cavity of the suction tube for abortion connects with the suction device and the first conduit connects with another gas conveying device, and wherein the outlet of the first cavity of the suction tube for abortion connects with the fluid connection of the suction device through a handle, and the first conduit connects with the fluid connection of another gas conveying device through a handle.

12. The suction tube to claim 11, wherein a visual guiding device is provided, wherein the visual guiding device is provided near the inlet of the first cavity, and the first conduit and the second conduit are in the rear of the visual guiding device.

13. The suction tube to claim 12, wherein the visual guiding device includes a pin-type camera and a light source; and wherein the fluid includes gas, liquid, or a mixture of liquid and gas.

14. The suction tube to claim 12, wherein the first conduit is a pressure reducing outlet pipe, and the second conduit is a negative pressure inlet pipe.

15. The suction tube to claim 12, wherein the suction tube further comprising a handle, and wherein the handle includes a major joint for connecting with the suction tube body, an inlet for connecting with the suction outlet of the suction cavity, and a circulation channel between them; and the handle further includes a one-way outlet and a one-way inlet; the one-way inlet connects with the negative pressure inlet pipe, wherein a one-way air inlet valve is provided, and the one-way outlet connects with the pressure reducing outlet pipe, wherein a one-way air outlet valve is provided.
